(19) United States
(12) Patent Application Publication
(10) Pub. No.: US 2008/0177181 A1
(43) Pub. Date: Jul. 24, 2008

(54) SYNCHRONIZING ULTRASOUND AND ECG DATA

(76) Inventor: Harold M. Hastings, Garden City, NY (US)

Correspondence Address: PROSKAUER ROSE LLP;PATENT DEPARTMENT, 1585 BROADWAY, NEW YORK, NY, 10036-8299, US

(21) Appl. No.: 12/018,977

(22) Filed: Jan. 24, 2008

Related U.S. Application Data
(60) Provisional application No. 60/886,483, filed on Jan. 24, 2007.

(51) Int. Cl. A61B 8/00

(52) U.S. Cl. 600/440

(57) ABSTRACT

ECG data and ultrasound data are synchronized by adding a marker to both sets of data, then detecting the position of the markers in the data, and using the detected positions to align the two sets of data in time. This enables an operator to visualize which frame of ultrasound data corresponds in time to which portion of the ECG waveform.

### CROSS REFERENCE TO RELATED APPLICATIONS

This application claims the benefit of U.S. provisional application No. 60/886,483, filed Jan. 24, 2007, which is incorporated herein by reference.

### BACKGROUND

The operation of the heart of a patient can be monitored in vivo using a variety of approaches. One commonly used approach for monitoring the operation of the heart is an electrocardiogram (ECG or EKG) which is a graphical representation of the electrical activity of the heart over time. Another commonly used approach for monitoring the operation of the heart is the echocardiogram, which is typically used to generate a two dimensional moving video image of the heart in real time, while the heart is beating. Each of these approaches provides a different set of information about the operation of the heart, in real time.

### SUMMARY

ECG data and ultrasound data are synchronized by adding a marker to both sets of data, then detecting the position of the markers in the data, and using the detected positions to align the two sets of data in time. This enables an operator to visualize which frame of ultrasound data corresponds in time to which portion of the ECG waveform.

### BRIEF DESCRIPTION OF THE DRAWINGS

FIG. 1 is a graph of a set of waveforms that are introduced into the ECG and ultrasound systems, to form the markers in the respective data.

FIG. 2 is a schematic diagram of a circuit that is suitable for generating the markers for the ECG and ultrasound systems.

FIG. 3 is a schematic diagram of an alternative circuit that is suitable for generating the markers for the ECG and ultrasound systems.

### DETAILED DESCRIPTION OF THE PREFERRED EMBODIMENTS

The inventor has recognized that significant advantages can be obtained by synchronizing ECG data and ultrasound data so that the operator can visualize which frame of ultrasound data corresponds in time to which portion of the ECG waveform, and the present invention relates to synchronizing or registering ECG and ultrasound data. While the primary intended application is in the field of cardiac ultrasound, it can be used in numerous other applications as well.

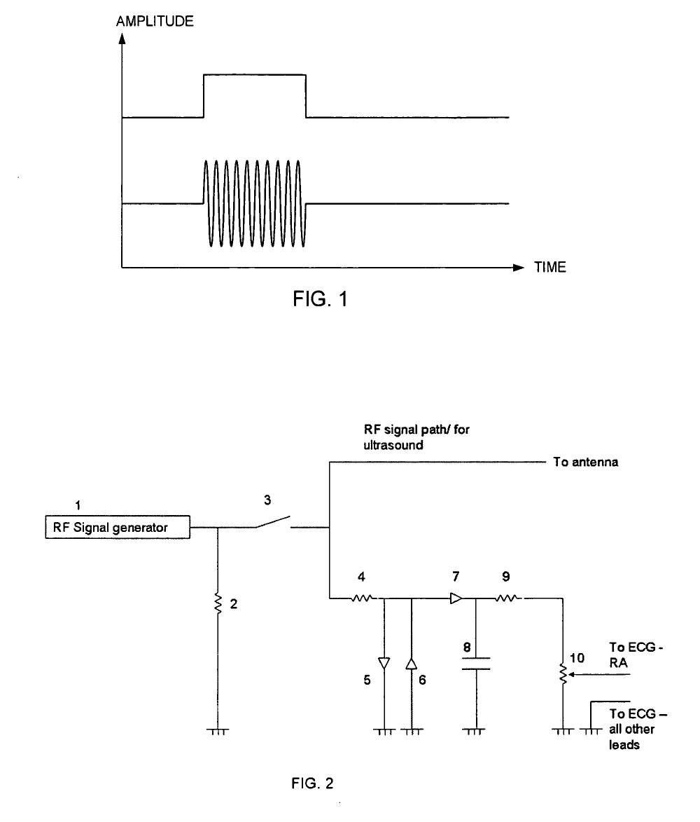

In one preferred implementation, synchronization is provided by simultaneously generating (a) a first signal that can be detected by an ECG machine; and (b) a second signal that can be detected by an ultrasound machine. One suitable set of such signals is depicted in FIG. 1, in which the top trace can be detected by an ECG machine and the bottom trace can be detected by an ultrasound machine.

For the signal that is detectable by the ECG machine, a short, positive-going pulse of current from the baseline for the ECG signal is suitable. For the signal that is detectable by the ultrasound machine, a short burst of ultrasound RF is suitable, with the RF carrier preferably close to the operating frequency of the transducer (e.g., 6 MHz). Preferably, the signal should be at least as long as the frame-to-frame interval of the ultrasound system. For example, a 20 mS pulse would be suitable for a system that is imaging at 50 frames per second. Preferably, the rise and fall times of the pulse should be much shorter than the frame-to-frame interval (e.g., less than 1 mS).

The burst of RF is synchronized with the pulse that is applied to the ECG machine, e.g., as shown in FIG. 1. Note that while FIG. 1 schematically depicts the lower waveform with ten cycles of the wave, many more cycles of the wave will be present in the waveforms that are actually used. For example, with a 6 MHz RF signal, there would be 120,000 cycles in a 20 mS burst.

The required synchronized pulses can be generated with the circuit depicted in FIG. 2. A commercially available function generator (e.g., BK Precision 4017A) may be used as the signal generator 1, in which case it should preferably be set to match the center frequency of the ultrasound transducer that is being used. For example, if the center frequency of the ultrasound transducer is 6 MHz, the signal generator 1 should be set to generate a 6 MHz sine wave. Alternatively, a sine wave oscillator may be custom designed to operate at the desired frequency, in which case the entire circuit can be battery operated, and optionally electrically isolated. Resistor 2 is a load resistor that preferably matches the output impedance of the signal generator (e.g., 50Ω).

Switch 3 may be a manually operated switch that, when actuated, passes the output of the signal generator 1 to the rest of the circuit. Alternatively, it may be an electronic switch that is closed for a predetermined period of time (e.g., between about 10 and about 50 mS, and preferably about 10 mS), under control of a suitable circuit (e.g., a one-shot).

Resistor 4 (e.g., 100Ω) and back to back diodes 5 and 6 form a protective circuit to limit the voltage that is applied to the ECG system. Diode 7 half-wave rectifies the AC, and capacitor 8 (e.g., 1000 pF) captures the peaks. The capacitor 8 is discharged through resistor 9 (e.g., 3 kΩ) and the track of potentiometer 10 (e.g., 10Ω), and the output voltage is set to match the ECG system by adjusting the position of the wiper of the potentiometer 10. Taken together, Diode 7, capacitor 8, resistor 9, and potentiometer 10 operate as an envelope detector.

To adjust the circuit for operation, the ultrasound box is set up for imaging, and the antenna is placed near the ultrasound probe or near the connector to the ultrasound box. (A physical connection to the ultrasound box is not required to couple the signal into the ultrasound box.) Closing switch 3 sends RF to the antenna, and the signal generator output level and/or antenna position is adjusted until the ultrasound image appears noticeable whiter than it was when the switch 3 was open. This increase in whiteness serves as a marker or artifact in the ultrasound image. Next, the output from the envelope detector is connected to the ECG machine. One suitable way to make this connection is by connecting the output from the envelope detector to the RA input of the ECG machine and connecting the ground to the other leads of the ECG machine, but other lead connection arrangements may also be used, as will be apparent to persons skilled in the relevant arts. Pulses are then generated by closing and opening the switch 3, and adjusting potentiometer 10 until a satisfactory signal appears on the ECG, in the form of the leading edge of a positive going pulse, analogous to the upstroke of an R-wave. This pulse serves as a marker or artifact in the electrocardiogram.

After the adjustment, the ultrasound system and the ECG system are operated simultaneously to capture images and an electrocardiogram of the subject. While this is happening, pulses may be generated by closing and opening S3, and the ultrasound images and the electrocardiogram are recorded. Afterwards, the ultrasound image is played back frame by frame, and the appearance of the marker in the ultrasound images is compared to the appearance of the marker in the electrocardiogram. A timing relationship between the ultrasound image and the electrocardiogram can then be determined. Alternatively, a pulse train generator may be used to generate pulses at regular intervals, and a timing relationship between the ultrasound image and the electrocardiogram may then be determined using the pulse repetition rate.

Once the timing relationship between the two systems has been established, it can be calculated for subsequent times by tracking the amount of time elapsed in both systems. Since only one frame of an ultrasound image is typically displayed at any given instant, and an electrocardiogram displays a number of seconds of data all at once, one suitable user interface for indicating the timing relationship between the ultrasound images and the electrocardiogram is to colorize the spot on the electrocardiogram trace that corresponds to the frame of ultrasound that is being displayed at any given instant. When the ultrasound image is played back, the colorized spot would then move along the electrocardiogram trace. Alternatively, if the current time corresponds to a fixed position on the electrocardiogram display screen, the fixed position could be marked on the screen using a vertical line, and the ultrasound frame that corresponds to whatever portion of the electrocardiogram is at that fixed position at any given instant could be displayed. A wide variety of alternative user interfaces for displaying both sets of information and indicating the timing relationship between them can be readily envisioned.

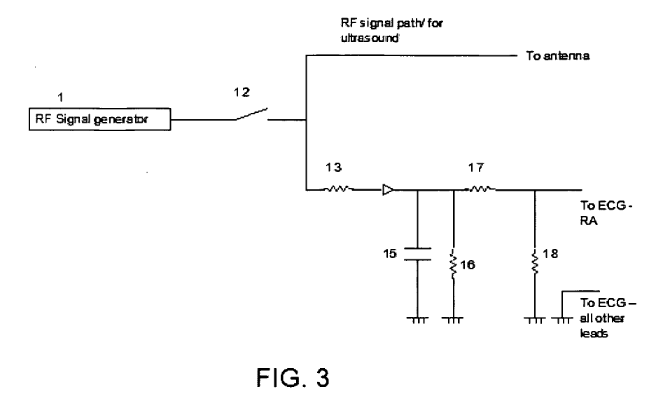

FIG. 3 is a schematic diagram of an alternative circuit for generating the markers in the ultrasound and the electrocardiogram. It is similar to the circuit shown in FIG. 2, except that the protection diodes 5 and 6 are omitted, and the combination of the resistor 9 and the potentiometer 10 (which did double-duty of both discharging the capacitor and dividing down the voltage) are replaced by a fixed resistor 16 for discharging the capacitor, and a pair of resistors 17 and 18 for dividing down the voltage. A suitable set of component values for this embodiment is: 200Ω for R13, 10 kΩ for R16, 220 kΩ for R17, 200Ω for R18, and 1000 pF for C15.

Synchronizing the ultrasound image with the ECG can be especially important in cardiac resynchronization therapy (CRT). In CRT one is interested the use of bi-ventricular pacemakers to overcome problems in the timing of cardiac wall movement—dyssynchrony. In particular, one would like to know that contraction is appropriately synchronous throughout the left ventricle—except for a smooth gradient from apex to base. Ultrasound data accurately timed with respect to the ECG and in particular the R-wave, for example, in the form of CINE loops, could be used to assess the appropriateness of the placement of pacemaker leads and the timing of pacemaker impulses.

I claim:

1. A method of synchronizing an electrocardiogram measurement with an ultrasound image, the method comprising the steps of:
obtaining an electrocardiogram of at least one beat of a subject's heart using an ECG system;
coupling, into the ECG system, a first signal that introduces a marker into the electrocardiogram;
obtaining an ultrasound image of the subject using an ultrasound system, wherein the ultrasound image includes enough frames to depict at least one beat of the subject's heart; and
coupling, into the ultrasound system, a second signal that introduces a marker into the ultrasound image, wherein the first signal and the second signal are coupled into the ECG system and the ultrasound system, respectively, at substantially the same time.

2. The method of claim 1, wherein the first signal comprises a rectangular pulse and the second signal comprises a burst of RF at a frequency that is detectable by the ultrasound system.

3. The method of claim 1, wherein the first signal comprises a rectangular pulse and the second signal comprises a burst of RF with a center frequency between about 4 and about 8 MHz, and the duration of both the first signal and the second signal is between about 10 and about 50 mS.

4. The method of claim 1, wherein the first signal comprises a rectangular pulse and the second signal comprises a burst of RF with a center frequency between about 4 and about 8 MHz, and the duration of both the first signal and the second signal is about 20 mS.

5. The method of claim 1, wherein the first signal comprises a plurality of rectangular pulses spaced at regular intervals, and the second signal comprises a plurality of bursts of RF at a frequency that is detectable by the ultrasound system, spaced at the regular intervals.

6. The method of claim 1, further comprising the steps of:
identifying at least one portion of the electrocardiogram in which the first marker appears;
identifying at least one frame of the ultrasound image that contains the second marker; and
aligning the electrocardiogram and the ultrasound image in time based on the identified at least one portion of the electrocardiogram and the identified at least one frame of the ultrasound image.

7. The method of claim 6, wherein the first signal comprises a rectangular pulse and the second signal comprises a burst of RF with a center frequency between about 4 and about 8 MHz, and the duration of both the first signal and the second signal is between about 10 and about 50 mS.

8. The method of claim 6, wherein the first signal comprises a rectangular pulse and the second signal comprises a burst of RF with a center frequency between about 4 and about 8 MHz, and the duration of both the first signal and the second signal is about 20 mS.

9. The method of claim 6, wherein the first signal comprises a plurality of rectangular pulses spaced at regular intervals, and the second signal comprises a plurality of bursts of RF at a frequency that is detectable by the ultrasound system, spaced at the regular intervals.

10. A method of synchronizing an electrical measurement with an ultrasound image, the method comprising the steps of:
obtaining an electrical measurement during at least one beat of a subject's heart using an electrical system;
coupling, into the electrical system, a first signal that introduces a marker into the electrical measurement;
obtaining an ultrasound image of the subject using an ultrasound system, wherein the ultrasound image includes enough frames to depict at least one beat of the subject's heart; and
coupling, into the ultrasound system, a second signal that introduces a marker into the ultrasound image, wherein a known time relationship exists between the first signal and the second signal.

11. The method of claim 10, wherein the electrical measurement comprises an electrocardiogram and the electrical system comprises an ECG system.

12. The method of claim 10, wherein the first signal and the second signal are substantially simultaneous.

13. The method of claim 10, wherein the first signal comprises a rectangular pulse and the second signal comprises a burst of RF at a frequency that is detectable by the ultrasound system.

14. The method of claim 10, wherein the first signal comprises a rectangular pulse and the second signal comprises a burst of RF with a center frequency between about 4 and about 8 MHz, and the duration of both the first signal and the second signal is between about 10 and about 50 mS.

15. The method of claim 10, wherein the first signal comprises a rectangular pulse and the second signal comprises a burst of RF with a center frequency between about 4 and about 8 MHz, and the duration of both the first signal and the second signal is about 20 mS.

16. The method of claim 10, further comprising the steps of:
identifying at least one portion of the electrical measurement in which the first marker appears;
identifying at least one frame of the ultrasound image that contains the second marker; and
aligning the electrical measurement and the ultrasound image in time based on (a) the identified at least one portion of the electrical measurement, (b) the identified at least one frame of the ultrasound image, and (c) the known time relationship.

17. The method of claim 16, wherein the first signal comprises a rectangular pulse and the second signal comprises a burst of RF with a center frequency between about 4 and about 8 MHz, and the duration of both the first signal and the second signal is between about 10 and about 50 mS.

18. The method of claim 16, wherein the first signal comprises a rectangular pulse and the second signal comprises a burst of RF with a center frequency between about 4 and about 8 MHz, and the duration of both the first signal and the second signal is about 20 mS.

19. An apparatus for synchronizing an electrocardiogram measurement with an ultrasound image comprising:
an input configured to accept an RF signal;
a switch operatively connected to output a burst of the RF signal upon actuation;
an antenna operatively connected to broadcast the burst of the RF signal; and
an envelope detector operatively connected to detect an envelope of the burst of the RF signal and generate an output corresponding to the detected envelope, with an output level that is suitable for coupling into an ECG system.

20. The apparatus of claim 19, wherein the RF signal has a center frequency between about 4 and about 8 MHz.
